(19) United States
(12) Patent Application Publication
(10) Pub. No.: US 2024/0108397 A1
(43) Pub. Date: Apr. 4, 2024

(54) SURGICAL TOOLS WITH NESTED PULLEYS

(71) Applicant: Cilag GmbH International, Zug (CH)

(72) Inventors: Austin E. Wise, Cincinnati, OH (US); Konstantin G. Zabotkin, Mason, OH (US)

(21) Appl. No.: 17/956,074

(22) Filed: Sep. 29, 2022

(51) Int. Cl. A61B 18/14; A61B 34/00; A61B 34/30

(52) U.S. Cl. CPC A61B 18/1445; A61B 18/1482; A61B 34/30; A61B 34/71; A61B 2018/00595

(57) ABSTRACT

Surgical tools that are capable of a higher mechanical advantage without increasing the overall width of the tool. The surgical tool includes a wrist that may comprise a first pulley adjacent to a second pulley, the first pulley may have a first lip and a second lip, and the second pulley may have a first lip and second lip. The first lip of the second pulley overlaps the second lip of the first pulley and at least a portion of the first lip of the second pulley is recessed within a body of the first pulley.

### TECHNICAL FIELD

The systems and methods disclosed herein are directed to robotic surgical tools and, more particularly to, articulable surgical tools.

### BACKGROUND

Minimally invasive surgical (MIS) tools and procedures can often be preferred over traditional open surgical techniques due to their ability to decrease post-operative recovery time and to leave minimal scarring. Laparoscopic surgery is one type of MIS procedure in which one or more small incisions are formed in the abdomen of a patient and a trocar is inserted through each incision to provide a surgical access pathway for an appropriate surgical tool. Trocars can additionally provide an internal seal assembly for maintaining insufflation of the abdomen during a surgical procedure.

A variety of MIS tools can be inserted into the abdominal cavity of a patient via a trocar and maneuvered from outside the abdomen. Laparoscopic surgical tools, for example, are often similar to those used in traditional surgical procedures, with the exception that laparoscopic surgical tools possess an elongate shaft extending from an end effector to a location outside the abdomen. The end effector is the surgically functional part of the surgical tool. The elongate shaft protrudes externally through a trocar when the surgical tool is inserted in the abdomen of a patient, and an external portion of the surgical tool provides a means for manipulating and communicating with the end effector. Once inserted in a patient's body, the end effector can engage and/or treat tissue in a number of ways to achieve a desired diagnostic or therapeutic effect. Illustrative end effectors of laparoscopic and similar surgical tools include, for example, scissors, graspers, needle drivers, clamps, staplers, cauterizers, suction tools, irrigation tools, and clip-appliers.

Robotic surgery represents a specialized class of laparoscopic surgical procedures. Instead of directly engaging a surgical tool as in traditional laparoscopic surgery, a surgeon instead manipulates and engages the surgical tool using an electronic interface communicatively coupled to a robotic manipulator. Manipulation and engagement of a surgical tool under robotic control can allow much more precise surgical procedures to be performed in many instances. A surgeon need not necessarily even be in the operating room with the patient. Advantageously, robotic surgical systems can allow intuitive hand movements to be realized by maintaining a natural eye-hand axis. In addition, robotic surgical systems can incorporate a “wrist” coupling the end effector to the elongate shaft to provide natural, hand-like articulation during a robotic surgical procedure. The wrist can also facilitate an expanded and more complex range of motion than is possible with a human wrist, which can allow highly elaborate and precise surgical procedures to be performed.

Many laparoscopic and robotic surgical tools utilize a wrist that contains a series of pulleys and drive cables to articulate the wrist and actuate the end effector. The drive cables are routed around the pulleys and finally terminated either on the wrist or the end effector. Depending on tool architectures, some cables can continue to wrap around the wrist and continue to be routed proximally; within these tools there is typically a crimp or other restraining feature in the wrist or end-effector. The routing of the cables around the pulleys creates a mechanical advantage and reduces cable friction when operating the tool. During a surgical procedure, different tasks will place different load demands on the cables. The higher the load on the cables, the more the cables wear and consequently the lower the expected use life of the cables. The wear of the cables can be reduced when the mechanical advantage gained by the pulleys in the wrist is increased. However, to increase mechanical advantage gained through the pulleys the cable is subjected to a higher bending angle known as a fleet angle that it must follow around the pulleys. A higher fleet angle increases the friction on the cables and can also lower the expected use life.

It is desired to have a surgical tool with a wrist that that increases mechanical advantage while also maintaining a low fleet angle.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 shows a diagram of an illustrative surgical tool that may incorporate certain principles of the present disclosure.

FIG. 2 shows a diagram illustrating the degrees of freedom through which a wrist of a surgical tool may articulate.

FIGS. 3-5 show various views of an illustrative surgical tool containing an end effector.

FIG. 6 shows a diagram illustrating coupling between a surgical tool and a robotic manipulator.

FIG. 7 shows an isolated view of a pulley arrangement of a surgical tool.

FIG. 8 shows an illustrated fleet angle of an elongated member,

FIG. 9 shows an isolated view of a pulley arrangement of a surgical tool.

FIGS. 10 and 11 shows a cross-sectional view of a pair of pulleys of surgical tool.

### DETAILED DESCRIPTION

The present disclosure generally describes surgical tools having an end effector operatively coupled to an elongate shaft and, more specifically, surgical tools and end effectors that may utilize elongated members and pulleys for articulation or actuation of the end effector.

Unwanted or premature failure of an elongated member on surgical tool during a procedure can be problematic. The present disclosure describes surgical tools and end effectors that are configured to perform multiple aspects of a surgical procedure, but with a significantly lower risk of experiencing a failure of an elongated member. More specifically, the present disclosure describes surgical tools with a wrist that incorporates one or more nested pulleys, which, lower the load placed on an elongated member without a substantial increase in friction to said elongated member. As such, the surgical tools disclosed herein are much less susceptible to premature failure and may allow a greater number of surgical procedures to be performed with the same surgical tool.

Before discussing additional details of the surgical tools and end effectors of the present disclosure and methods for their use, a brief overview of laparoscopic and similar surgical tools and robotic surgical systems will be provided hereinafter in order for the embodiments of the present disclosure to be better understood.

The terms “proximal” and “distal” are defined herein relative to the location of engagement by a surgeon or a robotic manipulator. The term “proximal” refers to a position closer to the location of engagement (i.e., further away from a patient), and the term “distal” refers to a position more removed from the location of engagement (i.e., nearer to a patient). Moreover, directional terms such as above, below, upper, lower, upward, downward, left, right, and the like are used to describe relative position in the figures and thus should not be considered limiting.

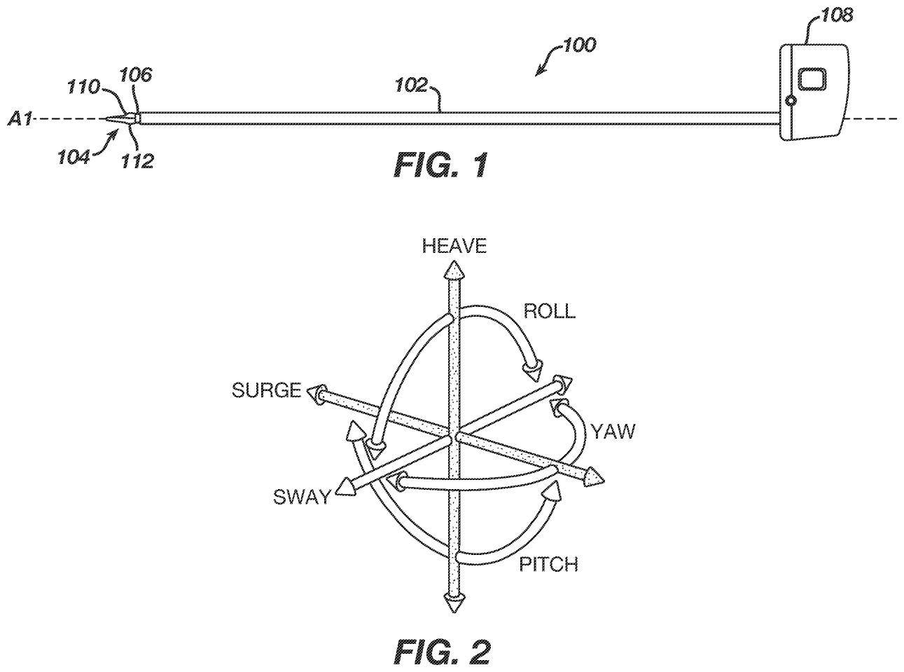

FIG. 1 shows a diagram of an illustrative surgical tool 100 that may incorporate certain principles of the present disclosure. Surgical tool 100 includes elongate shaft 102, end effector 104 located at a distal end of elongate shaft 102, and housing 108 located at a proximal end of elongate shaft 102. Wrist 106 is also located at a distal end of elongate shaft 102 and couples end effector 104 thereto. Housing 108 may be configured for releasable coupling with a mounting fixture of a robotic manipulator, alternately referred to as a “robot” or “surgical robot.” Housing 108 contains various mechanisms (obscured in FIG. 1) which may be actuated to produce one or more resultant motions in end effector 104. More particularly, actuation within housing 108 controls the operation of end effector 104 via retraction and extension of drive cables (or referred to as just “cables”) or similar elongate members (obscured in FIG. 1) that are operably engaged with end effector 104. Elongated members may include cables, rods, belts, wires, or strings made from any suitable metal (e.g., Nitinol, Tungsten, Stainless Steel, or Aluminum) or any suitable polymer (e.g., Polyester, Nylon, Polypropylene, Polyethylene, Aramids, or Carbon Fiber).

Housing 108 may be releasably coupled with the mounting fixture of a robotic manipulator in a variety of ways, such as by clamping or clipping thereto, or slidably mating therewith. Illustrative mechanisms for releasably coupling housing 108 to a mounting fixture are described in more detail in U.S. Patent Application Publication 2015/0209965, and U.S. Pat. Nos. 9,884,427; and 10,149,726; incorporated herein by reference in their entirety. Illustrative robotic surgical systems are also described in these references and in U.S. Pat. No. 8,831,782, which is also incorporated herein by reference in its entirety.

Continuing with FIG. 1, end effector 104 is configured to move relative to elongate shaft 102 at wrist 106, such as by pivoting at wrist 106, to position end effector 104 at a desired orientation and location relative to a surgical site during a surgical procedure. Housing 108 includes various components designed to position and operate various features of end effector 104 (e.g., one or more of clamping, firing, rotation, articulation, energy delivery, and the like). One or more elongate members extend from housing 108 through wrist 106 to facilitate articulation of end effector 104, as discussed in more detail herein. Elongate shaft 102 and end effector 104 coupled distally thereto may be configured to rotate about longitudinal axis A1. Various components of housing 108 can be configured to facilitate rotational motion of elongate shaft 102 and end effector 104 about longitudinal axis A1. Elongate shaft 102 may be fixed to housing 108, in which case surgical tool 100 may be rotated by the robotic manipulator to reposition elongate shaft 102 and end effector 104.

Surgical tool 100, particularly at end effector 104, can be configured to perform at least one surgical function. The choice of end effector 104 can determine which surgical function surgical tool 100 is able to perform. Illustrative configurations of end effector 104 that may be present in surgical tool 100 include, for example, forceps, graspers, needle drivers, scissors, electrocauterization tools that apply energy to tissue, staplers, clip appliers, suctioning tools, irrigation tools, imaging devices (e.g., endoscopes or ultrasonic probes), and any combination thereof. In at least one embodiment, surgical tool 100 may be configured to apply mechanical force to a tissue. The mechanical force can be conveyed to end effector 104 via the drive cables or similar elongate members extending through elongate shaft 102.

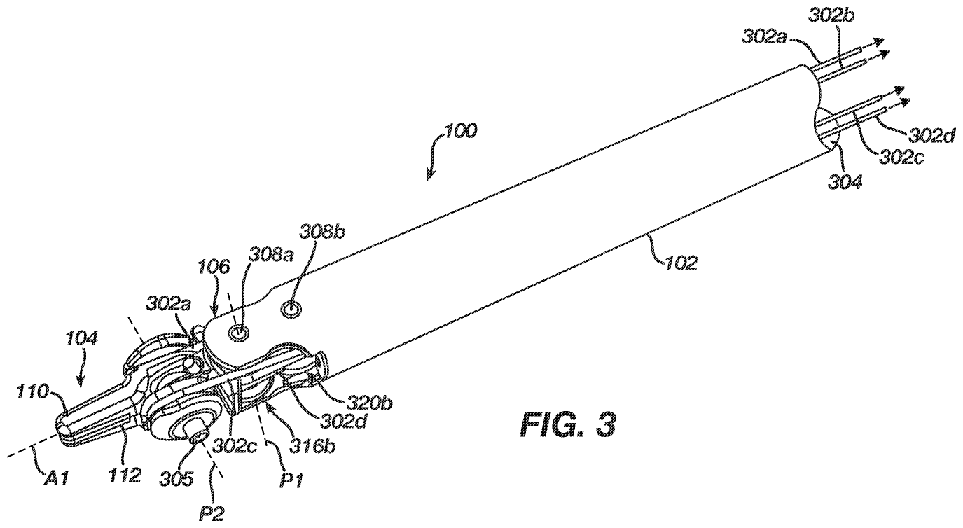

Elongate shaft 102 extends distally from housing 108 and has at least one lumen (see FIG. 3) extending internally therethrough. Elongate shaft 102 may be affixed to housing 108, but alternately may be releasably coupled so as to be interchangeable with other types of elongate shafts, such as elongate shafts having a differing diameter. Elongate shaft 102 may be rotatably coupled to housing 108.

End effector 104 can have a variety of sizes, shapes and configurations. In the illustrative configuration of FIG. 1, end effector 104 comprises a tissue grasper or needle driver having opposing jaws 110 and 112 that are configured to move (pivot) relative to one another between open and closed positions. In addition, the entirety of end effector 104 may pivot relative to elongate shaft 102 at wrist 106. Pivoting may place end effector 104 in a desired position to engage tissue or another surface during a surgical procedure.

Wrist 106 can likewise have a variety of configurations. In the illustrative configuration of FIG. 1, wrist 106 includes a joint configured to allow movement of end effector 104 relative to elongate shaft 102, such as a pivot joint at which jaws 110 and 112 are pivotally attached via a corresponding body. Illustrative configurations that may be similar to wrist 106 and are suitable for use in the embodiments of the present disclosure include those described in U.S. Pat. Nos. 9,884,427 and 10,149,726 and in and U.S. Patent Publication No. 2015/0025549, each previously incorporated by reference above.

FIG. 2 shows a diagram illustrating the degrees of freedom through which wrist 106 may articulate. More specifically, the degrees of freedom available to wrist 106 are represented by three translational or position variables (e.g., surge, heave and sway) and three rotational or orientation variables (e.g., Euler angles or roll, pitch and yaw). The translational and rotational variables collectively describe the position and orientation of one or more components of a surgical system (e.g., wrist 106 and associated end effector 104) with respect to a given frame of reference, such as a Cartesian coordinate system or spherical coordinate system. As illustrated in FIG. 2, the term “surge” refers to forward and backward movement, the term “heave” refers to up and down movement, and the term “sway” refers to left and right movement. With regard to the rotational terms in FIG. 2, “roll” refers to side-to-side tilting, “pitch” refers to forward and backward tilting, and “yaw” refers to left and right turning.

In some embodiments a pivoting motion can include pitch movement about a first axis of wrist 106 (e.g., X-axis), yaw movement about a second axis of wrist 106 (e.g., Y-axis), and combinations thereof to allow for 360° rotational movement of end effector 104 about wrist 106. In other embodiments a pivoting motion can be limited to movement in a single plane such that end effector 104 rotates only in a single plane (e.g., only pitch movement about a first axis of wrist 106 or only yaw movement about a second axis of wrist 106).

Surgical tool 100 includes a plurality of drive cables or similar elongate members (obscured in FIG. 1), which are configured to impart movement to end effector 104 relative to elongate shaft 102. Illustrative forms of the elongate members include, for example, cables, bands, lines, cords, wires, ropes, strings, twisted strings and the like. Elongate members can be formed from any of a variety of high-durability materials, such as a metal (e.g., tungsten, stainless steel, and like materials) or a polymer. One or more of the elongate members may be made of a flexible material. Illustrative cables and similar elongate members are described in U.S. Patent Application Publications 2015/0209965 and 2015/0025549, each previously incorporated herein by reference.

The disposition of the elongate members within surgical tool 100 is illustrated more fully in FIGS. 3-5, which show various enlarged views of elongate shaft 102, end effector 104, and wrist 106. Although surgical tool 100 is depicted as including four elongate members 302a-d, one pair being operatively coupled to each of jaws 110 and 112, alternative configurations can have differing numbers of elongate members. For example, a surgical tool having an end effector that does not require internal motion can include two elongate members configured to provide articulation upon longitudinal tensioning and de-tensioning.

As shown in FIGS. 3-5, elongate members 302a, 302b, 302c, and 302d extend longitudinally within lumen 304 of elongate shaft 102 through wrist 106 and operably engage end effector 104, as described hereinafter. The proximal ends of elongate members 302a-d are similarly operably engaged with components in housing 108 (not shown in FIGS. 3-5). One or more of elongate members 302a-d may be selectively translated longitudinally to cause end effector 104 to move (e.g., pivot in one or more locations) relative to elongate shaft 102. Depending on the required motion, one or more of elongate members 302a-d may translate longitudinally to articulate end effector 104 (e.g., to move jaws 110 and 112 at an angle in a same direction), to open end effector 104 (e.g., to move jaws 110 and 112 away from one another), to close end effector 104 (e.g., to move jaws 110 and 112 toward one another), or any combination thereof.

Although a single lumen 304 is depicted in FIG. 3, multiple lumens can be present in alternative embodiments, such that one or more of elongate members 302a-d is housed within each of the multiple lumens. In further alternative embodiments, one or more of elongate members 302a-d can extend along the exterior of elongate shaft 102, such as in longitudinal channels formed in an exterior surface of elongate shaft 102.

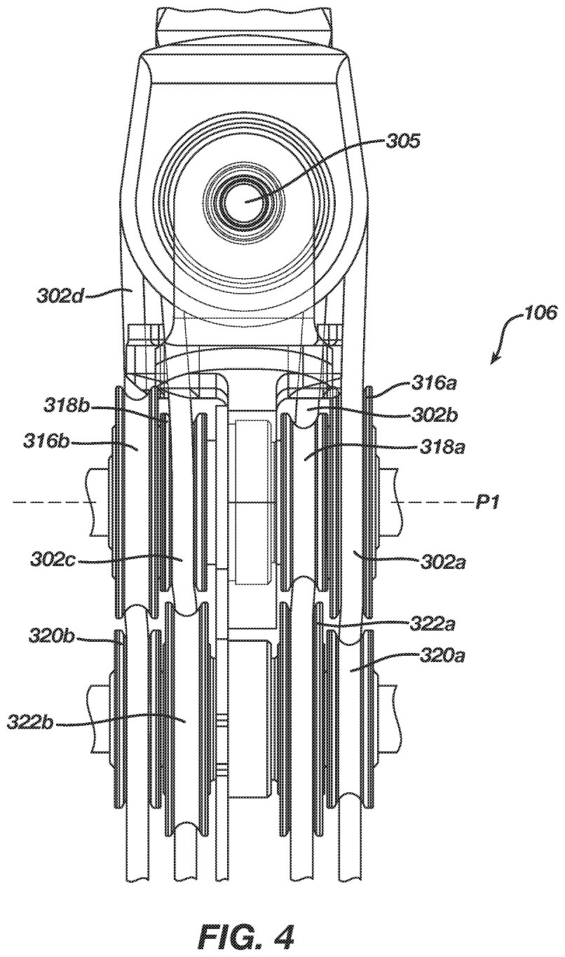

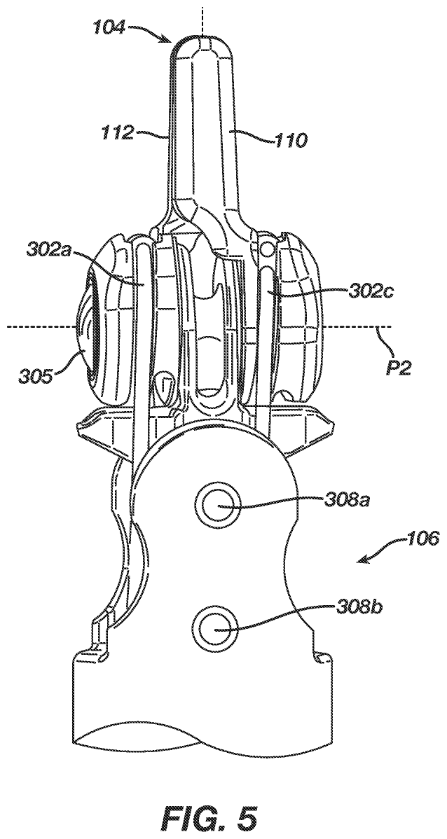

Referring still to FIG. 3, and with further reference to FIGS. 4 and 5, wrist 106 includes multiple pulleys for engaging and redirecting elongate members 302a-d during their longitudinal translation. Specifically, wrist 106 includes distal plurality of pulleys 316a, 316b, 318a and 318b, and proximal plurality of pulleys 320a, 320b, 322a and 322b. Clearance (best shown in FIG. 4) is provided between corresponding pulleys in the distal and proximal pluralities of pulleys, which is sized for passage of elongate members 302a-d therethrough. Pulleys 316a, 316b, 318a and 318b are mounted to distal wrist axle 308a, and pulleys 320a, 320b, 322a and 322b are mounted to proximal wrist axle 308b. End effector 104 is operably coupled to wrist 106 such that distal wrist axle 308a defines first pivot axis P1 during operation thereof.

Surgical tool 100 further includes second pivot axis P2 along end effector axle 305, about which jaws 110 and 112 are configured to pivot relative to each other from a closed position through a range of open positions, and/or about which jaws 110 and 112 are configured to move together during articulation of end effector 104. As illustrated, second pivot axis P2 is substantially perpendicular to longitudinal axis A1. Axes A1 and P2 may not be precisely perpendicular to one another but nevertheless be considered to be substantially perpendicular due to any number of factors, such as manufacturing tolerance and precision of measurement devices.

Surgical tool 100 may have two joints at second pivot axis P2, one joint for each of jaws 110 and 112. Actuation of at least one of elongate members 302a, 302b, 302c, and 302d causes movement of jaw 110 and/or jaw 112 at the associated joint(s) along second pivot axis P2. Jaws 110 and 112 may be configured to pivot in tandem at their associated joints. That is, during opening of jaws 110 and 112, each of jaws 110 and 112 rotates at its associated joint, and during closing of jaws 110 and 112, each of jaws 110 and 112 rotates in the opposite direction at its associated joint.

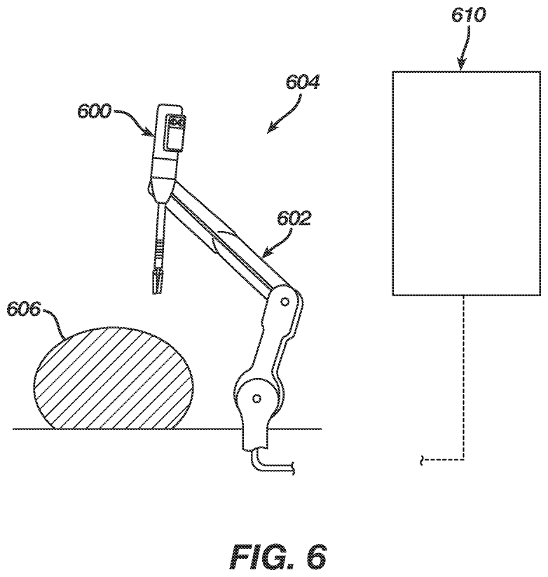

Surgical tool 100 may be configured for releasable coupling to a robotic manipulator. FIG. 6 shows a diagram illustrating coupling between a surgical tool and a robotic manipulator. It is to be understood that the manner of coupling depicted in FIG. 6 is illustrative in nature. In non-limiting variations, the type of surgical tool and/or robotic manipulator, and/or the manner of coupling, for example, may differ based upon considerations that will be familiar to one having ordinary skill in the art.

As depicted in FIG. 6, surgical tool 600 may be coupled to arm 602 of robotic manipulator 604. Robotic manipulator 604 and surgical tool 600 are positioned adjacent to patient 606 in order to conduct a surgical procedure thereon. Robotic manipulator 604 is in electronic communication with control system 610, through which a surgeon may move arm 602 and/or actuate surgical tool 600 according to one or more embodiments. Although FIG. 6 has depicted a wired connection between surgical tool 600 and control system 610, wireless configurations also reside within the scope of the present disclosure. Control system 610 may include vision control, processing control, or any combination thereof, using any combination of software and hardware implementation.

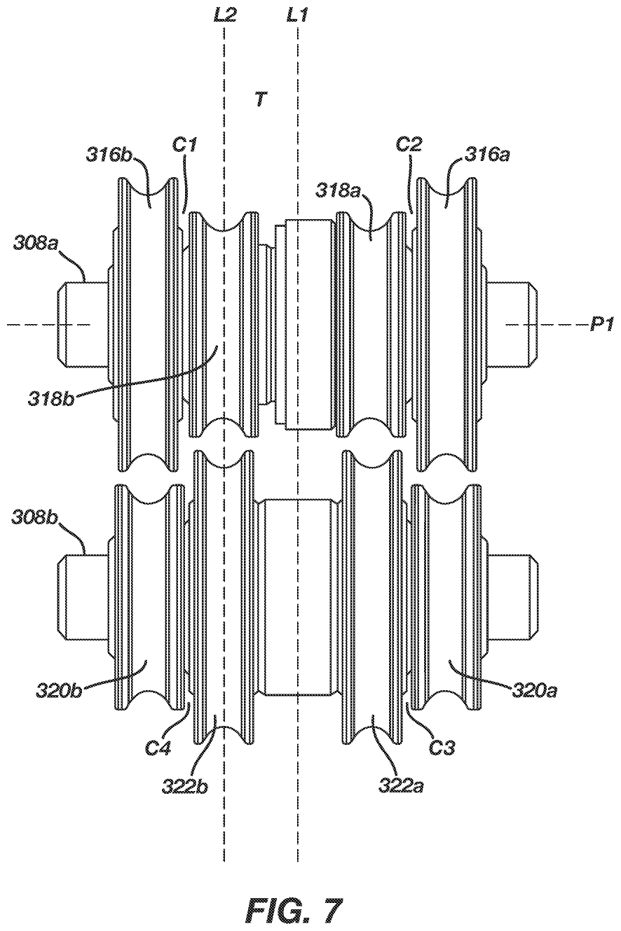

FIG. 7 is an isolated view of the pulleys within wrist 106. Shown are four pairs of pulleys, two distal pairs of pulleys that rotate on wrist axle 308a and two proximal pairs of pulleys that rotate on wrist axle 308b. Each of the pairs of pulleys has an outer pulley 316a, 316b, 320a, 320b, and an inner pulley 318a, 318b, 322a, and 322b. Between the respective outer and inner pulleys that form a pair is an offset or clearance C1, C2, C3 and C4 respectively. As the elongated members 302a, 302b, 302c, and 302d independently route their way around the pulleys of wrist 106, and end effector axle 305, the elongated members 302a, 302b, 302c, and 302d may be restrained from their natural position of rest about axle 305. The degree to which an elongated member is restrained from its natural position of rest is known as its “fleet angle”.

As described above, one or more of elongate members 302a, 302b, 302c, or 302d may be selectively translated longitudinally to cause end effector 104 to move (e.g., pivot in one or more locations) relative to elongate shaft 102. The pivot point for end effector 104 is the center axis of end effector axle 305. Longitudinal axis L1 extends proximally from the center axis of end effector axle 305. Longitudinal axis L2 extends both distally and proximally from the center line of pulley 318b. The distance between axis L1 and axis L2 defines a torque arm T. Torque arm T creates a mechanical advantage when operating elongated member 302C. Increasing the length of torque arm T, such as by increasing the distance between axis L1 and L2 can increase the mechanical advantage and thus lower the stress occurred in elongated member 302C. While it may be advantageous to increase torque arm T, it may result in increased friction between the elongated member and the pulley.

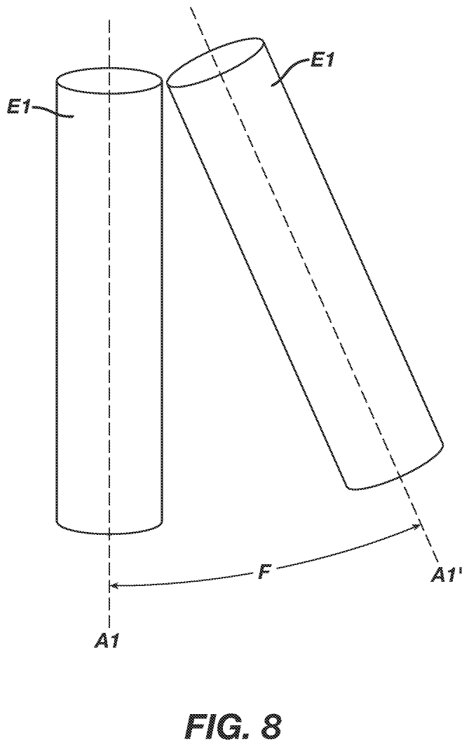

As best seen in FIG. 8 the elongate member E1 with center axis A1 hangs in its natural position of rest unrestrained. As elongate member E1 is forced away from its natural position of rest to its new position with center axis A1′ an angle F is formed between axis A1 and axis A1′. Angle F represents the fleet angle of the elongated member E1. An increase in angle F can increase friction of the elongated member E1 against the pulley or other mechanism restraining elongate member E1 from being in its natural position of rest. Increased friction on elongate member E1 can accelerate the wear and decrease the overall use life of elongate member E1.

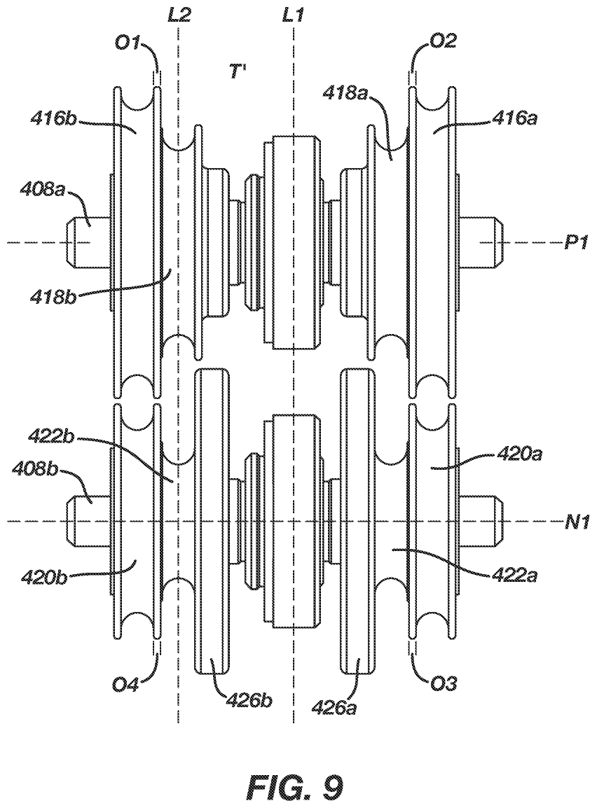

FIG. 9 shows an alternate pulley configuration for wrist 106. Wrist 106 has four pairs of pulleys, two distal pairs of pulleys that rotate on wrist axle 408a and two proximal pairs of pulleys that rotate on wrist axle 408b. Each of the pairs of pulleys has an outer pulley 416a, 416b, 420a, 420b, and an inner pulley 418a, 418b, 422a, and 422b. Between the respective outer and inner pulleys that form a pair is an overlap or nested region O1, O2, O3 and 04 respectively. Longitudinal axis L1 extends proximally from the center axis of end effector axle 305. Longitudinal axis L2′ extends both distally and proximally from the center line of pulley 418b. The distance between axis L1 and axis L2′ defines a torque arm T′. Inner pulley 418b has been shifted laterally from axis L1 while not increasing the overall width of wrist 106. As can be seen in FIG. 8, torque arm T′ is at a greater distance from L1 than torque arm T. Inner pulleys 422a, and 422b located on axis N1 are shown with an extended inner lip 426b and 426a respectively.

The fleet angles shown in FIG. 9. remain substantially similar as the fleet angles shown in FIG. 7. Nesting of the pulley pairs may increase the mechanical advantage of the elongated members without increasing the fleet angle and associated frictional stress. The nesting of the pulleys increases the torque arm from T to T′ while not increasing the overall width of the wrist or wrist axles 408a and 408b.

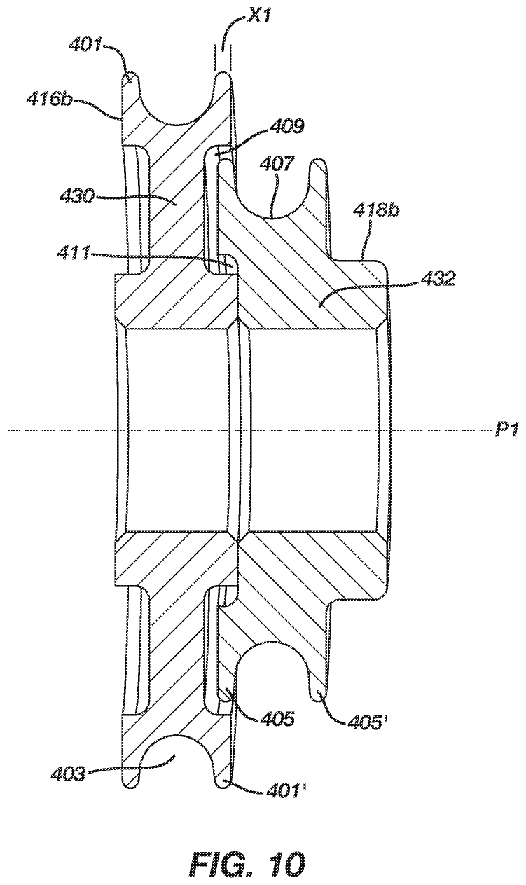

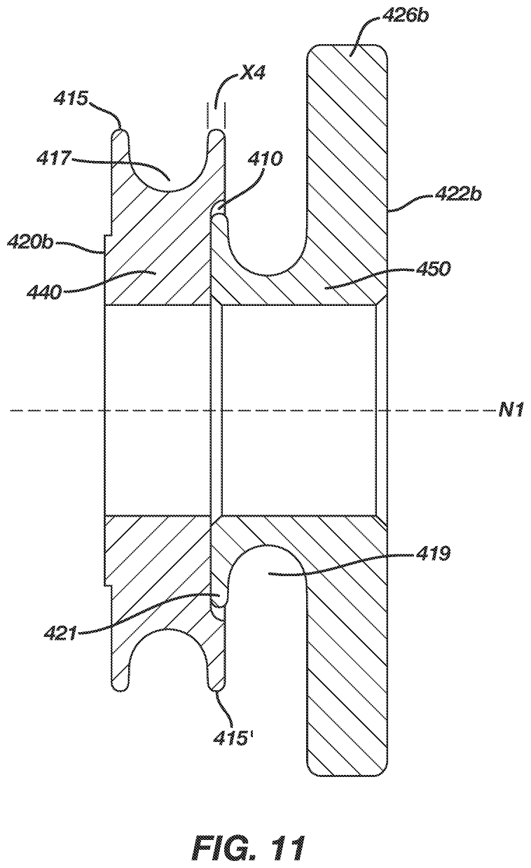

FIG. 10 is a cross section of a single pulley pair of wrist 106. Outer pulley 416b and inner pulley 418b of the shown pulley pair rotate independently about first pivot axis P1. Outer pulley 416b has an outer lip 401 and an inner lip 401′. Between outer lip 401 and inner lip 401′ is a center pocket 403. Center pocket 403 may be shaped and sized to accommodate or match that of the elongated member that pass through it. For example, center pocket 403 and 407 may be, but not limited to, a round, V-shape, flat, toothed, or other suitable profile. Additionally, wrist 106 may have more than one pulley, each pulley within wrist 106 may have a center pocket that is the same or different than the other pulleys in wrist 106. The outer lip 401 and inner lip 401′ of pulley 416b may have varying height relative to pocket 403 and be sized and shaped appropriately as to retain the elongated member in pocket 403. Likewise, inner pulley 418b may have an outer lip 405 and an inner lip 405′. Between outer lip 405 and inner lip 405′ is a center pocket 407. As described above, center pocket 407 may be shaped and sized to accommodate or match that of the elongated member that passes through it. The outer lip 405 and inner lip 405′ may have varying height relative to pocket 403 and may be sized and shaped appropriately as to retain the elongated member in pocket 407.

Still with reference to FIG. 10, the inner pulley 418b and outer pulley 416b may be nested or recessed into each other as to form an overlap region X1. Outer pulley 416b may include a channel 409 which may be U-shape, V-shape, or any other suitable shape formed in the pulley body 430 about axis P1. In FIG. 10, channel 409 may be formed on just one or both sides of pulley body 430. In the shown embodiment, channel 409 houses inner lip 405 of inner pulley 418b. Similarly, inner pulley 418b may include a recess 411 in pulley body 432 about axis P1 that houses a portion of outer pulley 416b. Overlap area X1 may vary in the amount of overlap from 1% of the width of the pulley lip up to the combined width of the pulley lip and channel minus the diameter of the elongated member the resides in the channel. Furthermore, lubricants may be used in channel 409 or recess 411 to minimize friction between inner pulley 418b and outer pulley 416b. Likewise inner pulley 418b and outer pulley 416b may themselves be made of a lubricious material such as PTFE, nylon, acetal, stainless steel, graphite, carbon fiber or other suitable low friction material.

FIG. 11 is a cross section of another representative embodiment which may include all or some of the features of the present disclosure. Outer pulley 420b and inner pulley 422b of the shown pulley pair rotate independently about axis N1. Outer pulley 420b has an outer lip 415 and an inner lip 415′. Between outer lip 415 and inner lip 415′ is a center pocket 417. Center pocket 417 may be shaped and sized to accommodate or match that of the elongated member that pass through it. For example, center pocket 417 and 419 may each independently be, but not limited to, either a round, V-shape, flat, toothed, or other suitable profile. Additionally, wrist 106 may have more than one pulley, each pulley within wrist 106 may have a center pocket that is the same or different than the other pulleys in wrist 106. The outer lip 415 and inner lip 415′ of pulley 420b may have varying height relative to pocket 417 and may be sized and shaped appropriately as to retain the elongated member in pocket 417. Likewise, inner pulley 422b may have an outer lip 421 and an inner lip 426b. Between outer lip 421 and inner lip 426b is a center pocket 419. As described above, center pocket 419 may be shaped and sized to accommodate or match that of the elongated member that pass through it. The outer lip 421 and inner lip 426b may have varying height relative to pocket 419 and be sized and shaped appropriately as to retain the elongated member in pocket 419. As shown in FIG. 11. inner lip 426b is extended to prevent derailment of the elongated member.

Further with reference to FIG. 11, the inner pulley 422b and outer pulley 420b may be nested or recessed into each other as to form an overlap region X4. Outer pulley 420b may include a recess 410 in pulley body 440 which may be U-shape, V-shape, or any other suitable shape formed about axis N1. Recess 410 houses inner lip 421 of inner pulley 422b. Similarly, inner pulley 422b may include a recess in pulley body 450 about axis N1 that houses a portion of outer pulley 420b. Overlap area X4 may vary in the amount of overlap. Furthermore, lubricants may be used in recess 410 to minimize friction between inner pulley 422b and outer pulley 420b. Likewise inner pulley 422b and outer pulley 420b may themselves be made of a lubricious material such as PTFE, nylon, acetal, stainless steel, graphite, carbon fiber or other suitable low friction material.

The number and location of pulleys in the device may vary as desired. More or less pullies may be used as desired, and each pulley may be located in a different area. Further, where pulleys are only shown inside wrist 106 it is understood and within the scope of this disclosure that the pulleys may be outside of wrist 106, for example but not limited to being within housing 108, elongated shaft 102, or end effector 104 without departure from this disclosure.

Aspects of the present disclosure may be integrated into a robotically enabled medical system capable of performing a variety of medical procedures, including both minimally invasive (e.g., laparoscopy) and non-invasive (e.g., endoscopy) procedures. Among exemplary endoscopy procedures, the system may be capable of performing bronchoscopy, ureteroscopy, and gastroscopy.

Implementations disclosed herein provide systems, methods and apparatus for instruments for use with robotic systems. It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

As used herein, the term “overlap” is intended to describe a spatial relation between components and not limiting to whether a component is overlying or underlying another component.

As used herein, the terms “generally” and “substantially” are intended to encompass structural or numeral modification, which do not significantly affect the purpose of the element or number modified by such term.

The innovations discussed herein include various aspects, including one or more of the following:
A. A surgical tool that may comprise: an elongate shaft having a proximal end and a distal end; and a housing coupled to the proximal end of the elongated shaft; and a wrist coupled to the distal end of the elongated shaft; and an end effector coupled to the wrist, where the wrist comprises: a first pulley adjacent to a second pulley; and wherein the first pulley has a first lip and a second lip; and wherein the second pulley has a first lip and second lip; and wherein the first lip of the second pulley overlaps the second lip of the first pulley and at least a portion of the first lip of the second pulley is recessed within a body of the first pulley.
B. A surgical tool that may comprise: an elongate shaft having a proximal end and a distal end; and a housing coupled to the proximal end of the elongated shaft; and an end effector coupled to the distal end of the elongated shaft, wherein the elongated shaft comprises: a first pulley adjacent to a second pulley; and wherein the first pulley has a first lip and a second lip; and wherein the second pulley has a first lip and second lip; and wherein the first lip of the second pulley overlaps the second lip of the first pulley; and wherein at least a portion of the first lip of the second pulley is recessed within a body of the first pulley.
C. A surgical tool that may comprise: an elongate shaft having a proximal end and a distal end; and a housing coupled to the proximal end of the elongated shaft; and a wrist coupled to the distal end of the elongated shaft; and an end effector coupled to the wrist, where the wrist comprises: a first pulley adjacent to a second pulley; and wherein the first pulley has a first lip and a second lip; and wherein the second pulley has a first lip and second lip; and wherein at least a portion of the first lip of the second pulley is recessed within a body of the first pulley so that the second lip of the first pulley becomes an extension of the first lip of the second pulley.

Each of the surgical tool described above as A, B, and C may each independently have one or more of the following additional elements in any combination:
Element 1: wherein the housing is configured for releasable engagement with a robotic surgical system.
Element 2: wherein the end effector delivers electrical energy.
Element 3: wherein the wrist can move the end effector in pitch and yaw degrees of freedom.
Element 4: wherein the second lip of the second pulley is radially extended more than the second lip of the first pulley.
Element 5: wherein the first pulley has a recess formed in the body about a rotational axis of the first pulley.
Element 6: wherein the recess is a channel.
Element 7: wherein the first lip of the second pulley, at least, partially occupies the channel.
Element 8: wherein the recess contains a lubricant
Element 9: wherein the second lip of the second pulley is radially extended more than the second lip of the first pulley.

By way of non-limiting example, combinations applicable to A, B, and C include: the surgical tool of A, B, or C in combination with elements 1 and 2; 1, 2 and 3; 1 and 4, 1, 4 and 5, 1 and 4-6, 1 and 7; 1, 7 and 8; 1 and 8; 1 and 9; 2 and 3; 2 and 4; 2, 4 and 5; 2, 4 and 6; 2 and 7; 2, 7 and 8; 2 and 8; 2 and 9; 3 and 4; 3 and 5, 3 and 6; 3 and 7; 3 and 8; 3 and 9; 4 and 5; 4 and 6; 4-6; 4 and 7; 4 and 8; 4 and 9; 7 and 8; 7 and 9; and 8 and 9.

Unless otherwise indicated, all numbers expressing quantities and the like in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

While various systems, tools and methods are described herein in terms of “comprising” various components or steps, the systems, tools and methods can also “consist essentially of” or “consist of” the various components and steps.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is the following:

1. A surgical tool comprising:
an elongated shaft having a proximal end and a distal end;
a housing coupled to the proximal end of the elongated shaft;
a wrist coupled to the distal end of the elongated shaft;
an end effector coupled to the wrist, where the wrist comprises:
a first pulley adjacent to a second pulley;
wherein the first pulley has a first lip and a second lip;
wherein the second pulley has a first lip and second lip; and
wherein the first lip of the second pulley overlaps the second lip of the first pulley and at least a portion of the first lip of the second pulley is recessed within a body of the first pulley.

2. The surgical tool of claim 1, wherein the housing is configured for releasable engagement with a robotic surgical system.

3. The surgical tool of claim 1, wherein the end effector delivers electrical energy.

4. The surgical tool of claim 1, wherein the wrist can move the end effector in pitch and yaw degrees of freedom.

5. The surgical tool of claim 1, wherein the second lip of the second pulley is radially extended more than the second lip of the first pulley.

6. The surgical tool of claim 1, wherein the first pulley has a recess formed in the body about a rotational axis of the first pulley.

7. The surgical tool of claim 6, wherein the recess is a channel.

8. The surgical tool of claim 7, wherein the first lip of the second pulley, at least, partially occupies the channel.

9. The surgical tool of claim 6, wherein the recess contains a lubricant.

10. A surgical tool comprising:
an elongated shaft having a proximal end and a distal end;
a housing coupled to the proximal end of the elongated shaft;
an end effector coupled to the distal end of the elongated shaft, wherein the elongated shaft comprises:
a first pulley adjacent to a second pulley;
wherein the first pulley has a first lip and a second lip;
wherein the second pulley has a first lip and second lip;
wherein the first lip of the second pulley overlaps the second lip of the first pulley; and
wherein at least a portion of the first lip of the second pulley is recessed within a body of the first pulley.

11. The surgical tool of claim 10, wherein the housing is configured for releasable engagement with a robotic surgical system.

12. The surgical tool of claim 10, wherein the end effector delivers electrical energy.

13. The surgical tool of claim 10, wherein the end effector can move in two degrees of freedom about the elongated shaft.

14. The surgical tool of claim 10, wherein the second lip of the second pulley is radially extended more than the second lip of the first pulley.

15. The surgical tool of claim 10, wherein the first pulley has a recess formed in the body about a rotational axis of the first pulley.

16. The surgical tool of claim 15, wherein the recess is a channel.

17. The surgical tool of claim 16, wherein the first lip of the second pulley, at least, partially occupies the channel.

18. The surgical tool of claim 15, wherein the recess contains a lubricant.

19. A surgical tool comprising:
an elongated shaft having a proximal end and a distal end;
a housing coupled to the proximal end of the elongated shaft;
a wrist coupled to the distal end of the elongated shaft;
an end effector coupled to the wrist, where the wrist comprises:
a first pulley adjacent to a second pulley;
wherein the first pulley has a first lip and a second lip;
wherein the second pulley has a first lip and second lip; and
wherein at least a portion of the first lip of the second pulley is recessed within a body of the first pulley so that the second lip of the first pulley becomes an extension of the first lip of the second pulley.

20. The surgical tool of claim 19, wherein the second lip of the second pulley is radially extended more than the second lip of the first pulley.
